(19) United States
(12) Patent Application Publication
(10) Pub. No.: US 2017/0143311 A1
(43) Pub. Date: May 25, 2017

(54) SYSTEMS AND METHODS FOR PULSED WAVE PREDICTIVE PROCESSING

(71) Applicant: EDAN INSTRUMENTS, INC., Shenzhen (CN)

(72) Inventors: Seshadri Srinivasan, Sunnyvale, CA (US); Yu Shangchong, Shenzhen (CN); Ling Feng, Shenzhen (CN)

(73) Assignee: EDAN INSTRUMENTS, INC., Shenzhen (CN)

(21) Appl. No.: 15/348,764

(22) Filed: Nov. 10, 2016

Related U.S. Application Data
(60) Provisional application No. 62/254,679, filed on Nov. 12, 2015.

(51) Int. Cl. A61B 8/08; A61B 8/06; A61B 8/00; A61B 8/02

(52) U.S. Cl. CPC A61B 8/5223; A61B 8/02; A61B 8/06; A61B 8/4483; A61B 8/461; A61B 8/5284

(57) ABSTRACT

A system includes an ultrasound transducer, a processing circuit, and a display. The ultrasound transducer is configured to detect ultrasound information from a patient and output the ultrasound information. The ultrasound information represents blood flow of the patient. The processing circuit is configured to generate a first waveform by automatically tracing the ultrasound information, identify a plurality of prior heart cycles of the first waveform and a current heart cycle of the first waveform, predict a second waveform based on the plurality of prior heart cycles, and update a visual representation of the current heart cycle based on the second waveform. The display is configured to display the visual representation of the current heart cycle.

### CROSS-REFERENCE TO RELATED APPLICATION

The present application claims the benefit of and priority to U.S. Provisional Application No. 62/254,679, titled “Pulsed Wave Predictive Processing,” filed Nov. 12, 2015, the disclosure of which is incorporated herein in its entirety for all purposes.

### TECHNICAL FIELD

The present disclosure generally relates to ultrasound systems. In some implementations, the present disclosure relates to ultrasound systems that can perform predictive processing of ultrasound data samples for displaying ultrasound spectra and images.

### BACKGROUND

Ultrasound systems can be used to detect information regarding a patient, in order to display such information to a medical professional or other user so that the user can make medical decisions based on the information. For example, an ultrasound transducer can transmit ultrasound waves into a body of the patient and detect return waves that may have been modified by blood flow and other structural features of the body of the patient, and a computer can communicate with the ultrasound transducer to received ultrasound information from the ultrasound transducer and display spectra and/or images using the ultrasound information. However, existing ultrasound display systems may lack the ability to account for discrepancies between the ultrasound information received and the underlying biological information, such as gaps, movement that cannot be tracked, and a poor signal to noise ratio, making it difficult to display such information in an accurate and easily understood manner and thus making it difficult for the user to make medical decisions based on the information.

### SUMMARY

One embodiment relates to a system. The system includes an ultrasound transducer, a processing circuit, and a display. The ultrasound transducer is configured to detect ultrasound information from a patient and output the ultrasound information. The ultrasound information represents blood flow of the patient. The processing circuit is configured to generate a first waveform by automatically tracing the ultrasound information, identify a plurality of prior heart cycles of the first waveform and a current heart cycle of the first waveform, predict a second waveform based on the plurality of prior heart cycles, and update a visual representation of the current heart cycle based on the second waveform. The display is configured to display the visual representation of the current heart cycle.

Another embodiment relates to a method. The method includes receiving ultrasound information representing blood flow of a patient from an ultrasound transducer. The method include generating a first waveform by automatically tracing the ultrasound information. The method includes identifying a plurality of prior heart cycles of the first waveform and a current heart cycle of the first waveform. The method includes predicting a second waveform based on the plurality of prior heart cycles. The method includes updating a visual representation of the current heart cycle based on the second waveform. The method includes displaying the visual representation of the current heart cycle.

Another embodiment relates to an ultrasound device. The ultrasound device includes a processing circuit. The processing circuit is configured to receive ultrasound information from an ultrasound transducer, generate a first waveform by automatically tracing the ultrasound information, identify a plurality of prior heart cycles of the first waveform and a current heart cycle of the first waveform, predict a second waveform based on the plurality of prior heart cycles, and update a visual representation of the current heart cycle based on the second waveform.

### BRIEF DESCRIPTION OF THE DRAWINGS

FIG. 1A is a perspective view of an ultrasound system according to an illustrative embodiment.

FIG. 1B is a perspective view of components of an ultrasound system according to an illustrative embodiment.

FIG. 2 is a block diagram illustrating components of an ultrasound system according to an illustrative embodiment.

FIG. 3 is a block diagram illustrating components of a processing circuit of an ultrasound system according to an illustrative embodiment.

FIG. 4 is a schematic diagram of ultrasound data sample spectra according to an illustrative embodiment.

FIG. 5 is a flow chart of a method of predictively generating a visual representation of ultrasound information according to an illustrative embodiment.

### DETAILED DESCRIPTION

Before turning to the Figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

Referring to the Figures generally, an ultrasound system can include an ultrasound transducer, a processing circuit, and a display. The ultrasound transducer is configured to detect ultrasound information from a patient and output the ultrasound information as an ultrasound data sample. The ultrasound information represents blood flow of the patient. The processing circuit is configured to generate a first waveform by automatically tracing the ultrasound information, identify a plurality of prior heart cycles of the first waveform and a current heart cycle of the first waveform, predict a second waveform based on the plurality of prior heart cycles, and update a visual representation of the current heart cycle based on the second waveform. The display is configured to display the visual representation of the current heart cycle.

In existing systems, ultrasound devices may have low signal-to-noise ratio, be unable to account for gaps or other artefacts, have poor contrast between spectrum information representing an anatomy of a patient and background noise, or otherwise be unable to accurately represent the anatomy of the patient so that a medical professional performing a procedure using the ultrasound device can effectively analyze the ultrasound information to determine treatment strategies. Systems and methods in accordance with the present disclosure can advantageously improve the display of ultrasound information by predictively generating visual representations of the ultrasound information, even when gaps, movement of anatomical features, and other confounding factors are present in the ultrasound information. In some embodiments, information from current and prior heart-cycles is used to predict the location and strength of a Doppler signal, and accordingly perform pulsed wave enhancement. Systems and methods according to the present disclosure can improve operation of ultrasound systems by improving tracking of a region of interest, reducing gaps in simultaneous mode, and improving the aesthetics of the displayed pulsed wave information. A continuous trace of the Doppler envelope can be used to identify the appropriate time segment for filling in gaps in simultaneous mode.

### A. Ultrasound System

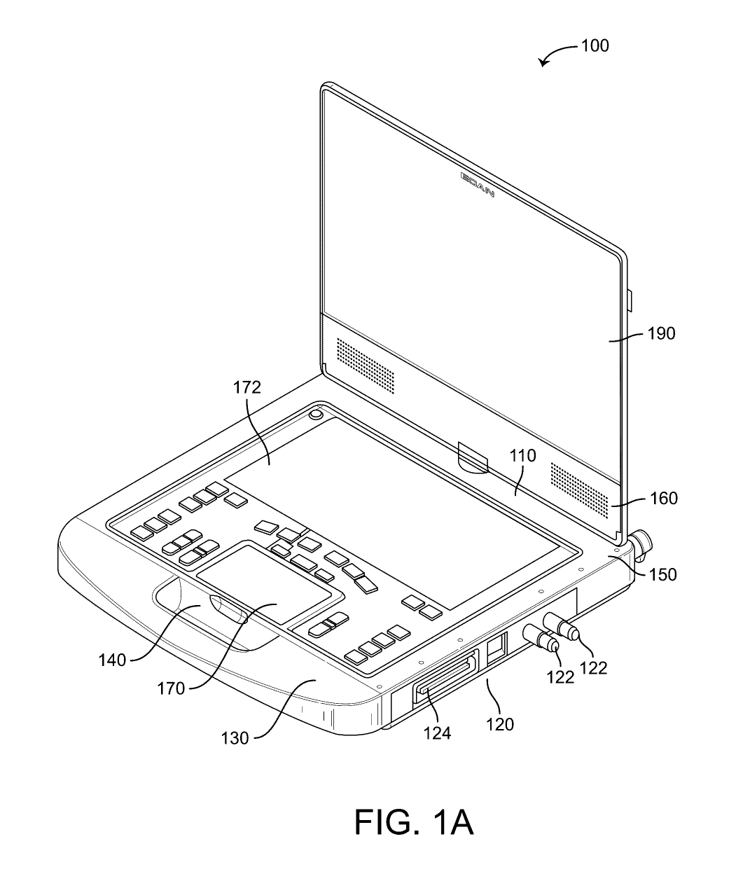

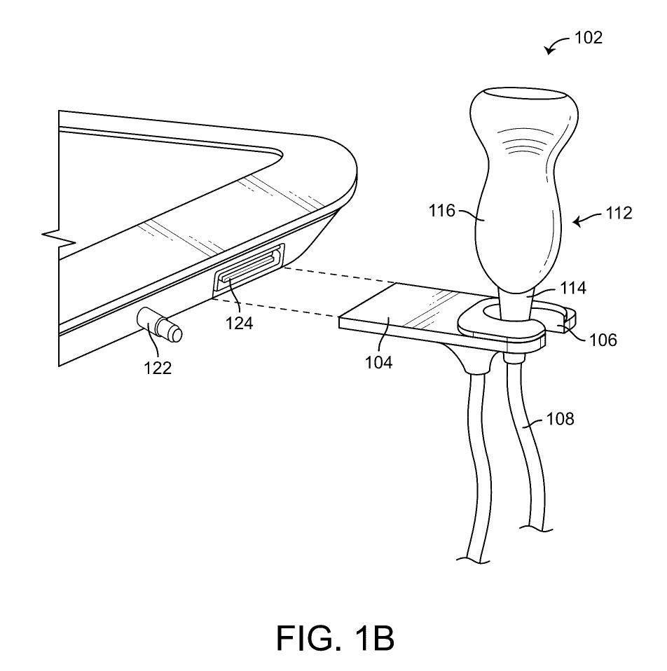

Referring now to FIG. 1A, one embodiment of portable ultrasound system 100 is illustrated. Portable ultrasound system 100 may include display support system 110 for increasing the durability of the display system. Portable ultrasound system 100 may further include locking lever system 120 for securing ultrasound probes and/or transducers. Some embodiments of portable ultrasound system 100 include ergonomic handle system 130 for increasing portability and usability. Further embodiments include status indicator system 140 which displays, to a user, information relevant to portable ultrasound system 100. Portable ultrasound system 100 may further include features such as an easy to operate and customizable user interface, adjustable feet, a backup battery, modular construction, cooling systems, etc.

Still referring to FIG. 1A, main housing 150 houses components of portable ultrasound system 100. In some embodiments, the components housed within main housing 150 include locking lever system 120, ergonomic handle system 130, and status indicator system 140. Main housing 150 may also be configured to support electronics modules which may be replaced and/or upgraded due to the modular construction of portable ultrasound system 100. In some embodiments, portable ultrasound system 100 includes display housing 160. Display housing 160 may include display support system 110. In some embodiments, portable ultrasound system 100 includes touchpad 170 for receiving user inputs and displaying information, touchscreen 172 for receiving user inputs and displaying information, and main screen 190 for displaying information.

Referring now to FIG. 1B, ultrasound transducer assembly 102 is shown. According to an exemplary embodiment, ultrasound transducer assembly 102 includes a connection assembly to pin (122) or socket (124) type ultrasound interface, shown as ultrasound interface connector 104, coupled to cable 108. Cable 108 may be coupled to a transducer probe 112. While FIG. 1B shows only one transducer assembly 102, more transducer assemblies may be coupled to the ultrasound system 100 based on the quantity of pin (122) or socket (124) type ultrasound interfaces.

Ultrasound interface connector 104 is movable between a removed position with respect to pin (122) or socket (124) type ultrasound interface, in which ultrasound interface connector 104 is not received by pin (122) or socket (124) type ultrasound interface, a partially connected position, in which ultrasound interface connector 104 is partially received by pin (122) or socket (124) type ultrasound interface, and a fully engaged position, in which ultrasound interface connector 104 is fully received by pin (122) or socket (124) type ultrasound interface in a manner that electrically couples transducer probe 112 to ultrasound system 100. In an exemplary embodiment, pin (122) or socket (124) type ultrasound interface may include a sensor or switch that detects the presence of the ultrasound interface connector 104.

In various exemplary embodiments contained herein, the ultrasound interface connector 104 may house passive or active electronic circuits for affecting the performance of the connected transducers. For example, in some embodiments the transducer assembly 102 may include filtering circuitry, processing circuitry, amplifiers, transformers, capacitors, batteries, failsafe circuits, or other electronics which may customize or facilitate the performance of the transducer and/or the overall ultrasound machine. In an exemplary embodiment, ultrasound interface connector 104 may include a bracket 106, where the transducer probe 112 may be stored when not in use.

Transducer probe 112 transmits and receives ultrasound signals that interact with the patient during the diagnostic ultrasound examination. The transducer probe 112 includes a first end 114 and a second end 116. The first end 114 of the transducer probe 112 may be coupled to cable 108. The first end 114 of the transducer probe 112 may vary in shape to properly facilitate the cable 108 and the second end 116. The second end 116 of the transducer probe 112 may vary in shape and size to facilitate the conduction of different types of ultrasound examinations. These first end 114 and second end 116 of transducer probe 112 variations may allow for better examination methods (e.g., contact, position, location, etc.).

A user (e.g., a sonographer, an ultrasound technologist, etc.) may remove a transducer probe 112 from a bracket 106 located on ultrasound interface connector 104, position transducer probe 112, and interact with main screen 190 to conduct the diagnostic ultrasound examination. Conducting the diagnostic ultrasound examination may include pressing transducer probe 112 against the patient's body or placing a variation of transducer probe 112 into the patient. The ultrasound spectrum or image acquired may be viewed on the main screen 190.

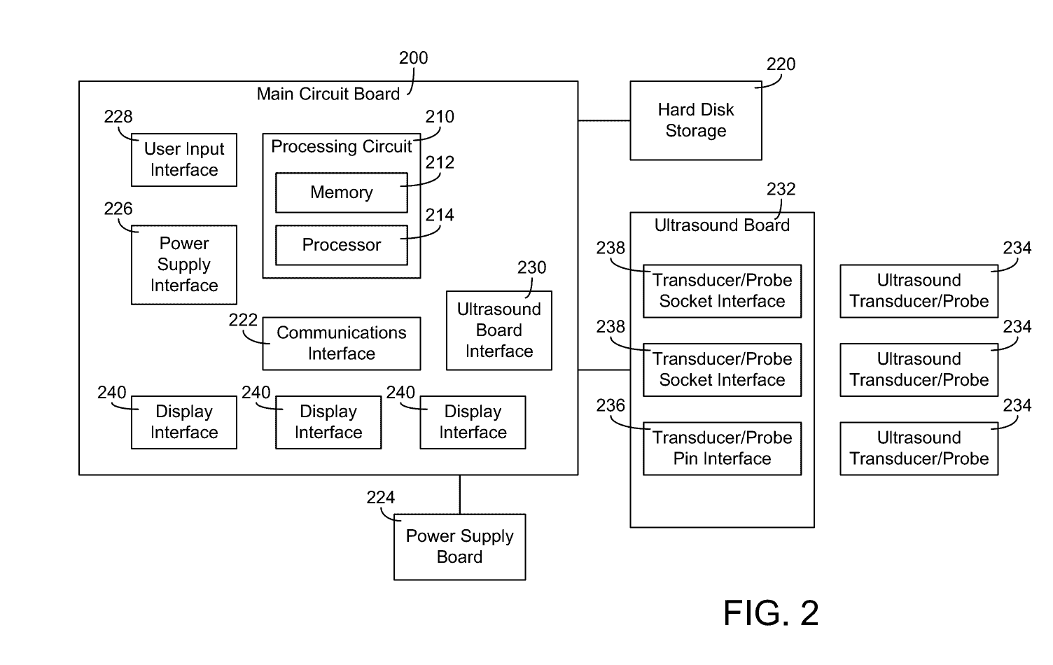

Referring to FIG. 2, a block diagram shows internal components of one embodiment of portable ultrasound system 100. Portable ultrasound system 100 includes main circuit board 200. Main circuit board 200 carries out computing tasks to support the functions of portable ultrasound system 100 and provides connection and communication between various components of portable ultrasound system 100. In some embodiments, main circuit board 200 is configured so as to be a replaceable and/or upgradable module.

To perform computational, control, and/or communication tasks, main circuit board 200 includes processing circuit 210. Processing circuit 210 is configured to perform general processing and to perform processing and computational tasks associated with specific functions of portable ultrasound system 100. For example, processing circuit 210 may perform calculations and/or operations related to producing a spectrum and/or an image from signals and/or data provided by ultrasound equipment, running an operating system for portable ultrasound system 100, receiving user inputs, etc. Processing circuit 210 may include memory 212 and processor 214 for use in processing tasks. For example, processing circuit 210 may perform calculations and/or operations.

Processor 214 may be, or may include, one or more microprocessors, application specific integrated circuits (ASICs), circuits containing one or more processing components, a group of distributed processing components, circuitry for supporting a microprocessor, or other hardware configured for processing. Processor 214 is configured to execute computer code. The computer code may be stored in memory 212 to complete and facilitate the activities described herein with respect to portable ultrasound system 100. In other embodiments, the computer code may be retrieved and provided to processor 214 from hard disk storage 220 or communications interface 222 (e.g., the computer code may be provided from a source external to main circuit board 200).

Memory 212 may be any volatile or non-volatile computer-readable storage medium capable of storing data or computer code relating to the activities described herein. For example, memory 212 may include modules which are computer code modules (e.g., executable code, object code, source code, script code, machine code, etc.) configured for execution by processor 214. Memory 212 may include computer code engines or circuits that can be similar to the computer code modules configured for execution by processor 214. Memory 212 may include computer executable code related to functions including ultrasound imagining, battery management, handling user inputs, displaying data, transmitting and receiving data using a wireless communication device, etc. In some embodiments, processing circuit 210 may represent a collection of multiple processing devices (e.g., multiple processors, etc.). In such cases, processor 214 represents the collective processors of the devices and memory 212 represents the collective storage devices of the devices. When executed by processor 214, processing circuit 210 is configured to complete the activities described herein as associated with portable ultrasound system 100, such as for predictively generating ultrasound spectra and/or images (e.g., for display by touchscreen 172 and/or display 190) based on predicting waveforms of the ultrasound information.

Hard disk storage 220 may be a part of memory 212 and/or used for non-volatile long term storage in portable ultrasound system 100. Hard disk storage 220 may store local files, temporary files, ultrasound spectra and/or images, patient data, an operating system, executable code, and any other data for supporting the activities of portable ultrasound device 100 described herein. In some embodiments, hard disk storage 220 is embedded on main circuit board 200. In other embodiments, hard disk storage 220 is located remote from main circuit board 200 and coupled thereto to allow for the transfer of data, electrical power, and/or control signals. Hard disk storage 220 may be an optical drive, magnetic drive, a solid state hard drive, flash memory, etc.

In some embodiments, main circuit board 200 includes communications interface 222. Communications interface 222 may include connections which enable communication between components of main circuit board 200 and communications hardware. For example, communications interface 222 may provide a connection between main circuit board 200 and a network device (e.g., a network card, a wireless transmitter/receiver, etc.). In further embodiments, communications interface 222 may include additional circuitry to support the functionality of attached communications hardware or to facilitate the transfer of data between communications hardware and main circuit board 200. In other embodiments, communications interface 222 may be a system on a chip (SOC) or other integrated system which allows for transmission of data and reception of data. In such a case, communications interface 222 may be coupled directly to main circuit board 200 as either a removable package or embedded package.

Some embodiments of portable ultrasound system 100 include power supply board 224. Power supply board 224 includes components and circuitry for delivering power to components and devices within and/or attached to portable ultrasound system 100. In some embodiments, power supply board 224 includes components for alternating current and direct current conversion, for transforming voltage, for delivering a steady power supply, etc. These components may include transformers, capacitors, modulators, etc. to perform the above functions. In further embodiments, power supply board 224 includes circuitry for determining the available power of a battery power source. In other embodiments, power supply board 224 may receive information regarding the available power of a battery power source from circuitry located remote from power supply board 224. For example, this circuitry may be included within a battery. In some embodiments, power supply board 224 includes circuitry for switching between power sources. For example, power supply board 224 may draw power from a backup battery while a main battery is switched. In further embodiments, power supply board 224 includes circuitry to operate as an uninterruptable power supply in conjunction with a backup battery. Power supply board 224 also includes a connection to main circuit board 200. This connection may allow power supply board 224 to send and receive information from main circuit board 200. For example, power supply board 224 may send information to main circuit board 200 allowing for the determination of remaining battery power. The connection to main circuit board 200 may also allow main circuit board 200 to send commands to power supply board 224. For example, main circuit board 200 may send a command to power supply board 224 to switch from one source of power to another (e.g., to switch to a backup battery while a main battery is switched). In some embodiments, power supply board 224 is configured to be a module. In such cases, power supply board 224 may be configured so as to be a replaceable and/or upgradable module. In some embodiments, power supply board 224 is or includes a power supply unit. The power supply unit may convert AC power to DC power for use in portable ultrasound system 100. The power supply may perform additional functions such as short circuit protection, overload protection, undervoltage protection, etc. The power supply may conform to ATX specification. In other embodiments, one or more of the above described functions may be carried out by main circuit board 200.

Main circuit board 200 may also include power supply interface 226 which facilitates the above described communication between power supply board 224 and main circuit board 200. Power supply interface 226 may include connections which enable communication between components of main circuit board 200 and power supply board 224. In further embodiments, power supply interface 226 includes additional circuitry to support the functionality of power supply board 224. For example, power supply interface 226 may include circuitry to facilitate the calculation of remaining battery power, manage switching between available power sources, etc. In other embodiments, the above described functions of power supply board 224 may be carried out by power supply interface 226. For example, power supply interface 226 may be a SOC or other integrated system. In such a case, power supply interface 226 may be coupled directly to main circuit board 200 as either a removable package or embedded package.

With continued reference to FIG. 2, some embodiments of main circuit board 200 include user input interface 228. User input interface 228 may include connections which enable communication between components of main circuit board 200 and user input device hardware. For example, user input interface 228 may provide a connection between main circuit board 200 and a capacitive touchscreen, resistive touchscreen, mouse, keyboard, buttons, and/or a controller for the proceeding. In one embodiment, user input interface 228 couples controllers for touchpad 170, touchscreen 172, and main screen 190 to main circuit board 200. In other embodiments, user input interface 228 includes controller circuitry for touchpad 170, touchscreen 172, and main screen 190. In some embodiments, main circuit board 200 includes a plurality of user input interfaces 228. For example, each user input interface 228 may be associated with a single input device (e.g., touchpad 170, touchscreen 172, a keyboard, buttons, etc.).

In further embodiments, user input interface 228 may include additional circuitry to support the functionality of attached user input hardware or to facilitate the transfer of data between user input hardware and main circuit board 200. For example, user input interface 228 may include controller circuitry so as to function as a touchscreen controller. User input interface 228 may also include circuitry for controlling haptic feedback devices associated with user input hardware. In other embodiments, user input interface 228 may be a SOC or other integrated system which allows for receiving user inputs or otherwise controlling user input hardware. In such a case, user input interface 228 may be coupled directly to main circuit board 200 as either a removable package or embedded package.

Main circuit board 200 may also include ultrasound board interface 230 which facilitates communication between ultrasound board 232 and main circuit board 200. Ultrasound board interface 230 may include connections which enable communication between components of main circuit board 200 and ultrasound board 232. In further embodiments, ultrasound board interface 230 includes additional circuitry to support the functionality of ultrasound board 232. For example, ultrasound board interface 230 may include circuitry to facilitate the calculation of parameters used in generating a spectrum and/or an image from ultrasound data provided by ultrasound board 232. In some embodiments, ultrasound board interface 230 is a SOC or other integrated system. In such a case, ultrasound board interface 230 may be coupled directly to main circuit board 200 as either a removable package or embedded package.

In other embodiments, ultrasound board interface 230 includes connections which facilitate use of a modular ultrasound board 232. Ultrasound board 232 may be a module (e.g., ultrasound module) capable of performing functions related to ultrasound imaging (e.g., multiplexing sensor signals from an ultrasound probe/transducer, controlling the frequency of ultrasonic waves produced by an ultrasound probe/transducer, etc.). The connections of ultrasound board interface 230 may facilitate replacement of ultrasound board 232 (e.g., to replace ultrasound board 232 with an upgraded board or a board for a different application). For example, ultrasound board interface 230 may include connections which assist in accurately aligning ultrasound board 232 and/or reducing the likelihood of damage to ultrasound board 232 during removal and/or attachment (e.g., by reducing the force required to connect and/or remove the board, by assisting, with a mechanical advantage, the connection and/or removal of the board, etc.).

In embodiments of portable ultrasound system 100 including ultrasound board 232, ultrasound board 232 includes components and circuitry for supporting ultrasound imaging functions of portable ultrasound system 100. In some embodiments, ultrasound board 232 includes integrated circuits, processors, and memory. Ultrasound board 232 may also include one or more transducer/probe socket interfaces 238. Transducer/probe socket interface 238 enables ultrasound transducer/probe 234 (e.g., a probe with a socket type connector) to interface with ultrasound board 232. For example, transducer/probe socket interface 238 may include circuitry and/or hardware connecting ultrasound transducer/probe 234 to ultrasound board 232 for the transfer of electrical power and/or data. Transducer/probe socket interface 238 may include hardware which locks ultrasound transducer/probe 234 into place (e.g., a slot which accepts a pin on ultrasound transducer/probe 234 when ultrasound transducer/probe 234 is rotated). In some embodiments, ultrasound board 232 includes two transducer/probe socket interfaces 238 to allow the connection of two socket type ultrasound transducers/probes 187.

In some embodiments, ultrasound board 232 also includes one or more transducer/probe pin interfaces 236. Transducer/probe pin interface 236 enables an ultrasound transducer/probe 234 with a pin type connector to interface with ultrasound board 232. Transducer/probe pin interface 236 may include circuitry and/or hardware connecting ultrasound transducer/probe 234 to ultrasound board 232 for the transfer of electrical power and/or data. Transducer/probe pin interface 236 may include hardware which locks ultrasound transducer/probe 234 into place. In some embodiments, ultrasound transducer/probe 234 is locked into place with locking lever system 120. In some embodiments, ultrasound board 232 includes more than one transducer/probe pin interfaces 236 to allow the connection of two or more pin type ultrasound transducers/probes 234. In such cases, portable ultrasound system 100 may include one or more locking lever systems 120. In further embodiments, ultrasound board 232 may include interfaces for additional types of transducer/probe connections.

With continued reference to FIG. 2, some embodiments of main circuit board 200 include display interface 240. Display interface 240 may include connections which enable communication between components of main circuit board 200 and display device hardware. For example, display interface 240 may provide a connection between main circuit board 200 and a liquid crystal display, a plasma display, a cathode ray tube display, a light emitting diode display, and/or a display controller or graphics processing unit for the proceeding or other types of display hardware. In some embodiments, the connection of display hardware to main circuit board 200 by display interface 240 allows a processor or dedicated graphics processing unit on main circuit board 200 to control and/or send data to display hardware. Display interface 240 may be configured to send display data to display device hardware in order to produce a spectrum and/or an image. In some embodiments, main circuit board 200 includes multiple display interfaces 240 for multiple display devices (e.g., three display interfaces 240 connect three displays to main circuit board 200). In other embodiments, one display interface 240 may connect and/or support multiple displays. In one embodiment, three display interfaces 240 couple touchpad 170, touchscreen 172, and main screen 190 to main circuit board 200.

In further embodiments, display interface 240 may include additional circuitry to support the functionality of attached display hardware or to facilitate the transfer of data between display hardware and main circuit board 200. For example, display interface 240 may include controller circuitry, a graphics processing unit, video display controller, etc. In some embodiments, display interface 240 may be a SOC or other integrated system which allows for displaying spectra and/or images with display hardware or otherwise controlling display hardware. Display interface 240 may be coupled directly to main circuit board 200 as either a removable package or embedded package. Processing circuit 210 in conjunction with one or more display interfaces 240 may display spectra and/or images on one or more of touchpad 170, touchscreen 172, and main screen 190.

Referring back to FIG. 1A, in some embodiments, portable ultrasound system 100 includes one or more pin type ultrasound probe interfaces 122. Pin type ultrasound interface 122 may allow an ultrasound probe to connect to an ultrasound board 232 included in ultrasound system 100. For example, an ultrasound probe connected to pin type ultrasound interface 122 may be connected to ultrasound board 232 via transducer/probe pin interface 236. In some embodiments, pin type ultrasound interface 122 allows communication between components of portable ultrasound system 100 and an ultrasound probe. For example, control signals may be provided to the ultrasound probe 112 (e.g., controlling the ultrasound emissions of the probe) and data may be received by ultrasound system 100 from the probe (e.g., imaging data).

In some embodiments, ultrasound system 100 may include locking lever system 120 for securing an ultrasound probe. For example, an ultrasound probe may be secured in pin type ultrasound probe interface 122 by locking lever system 120.

In further embodiments, ultrasound system 100 includes one or more socket type ultrasound probe interfaces 124. Socket type ultrasound probe interfaces 124 may allow a socket type ultrasound probe to connect to an ultrasound board 232 included in ultrasound system 100. For example, an ultrasound probe connected to socket type ultrasound probe interface 124 may be connected to ultrasound board 232 via transducer/probe socket interface 238. In some embodiments, socket type ultrasound probe interface 124 allows communication between components of portable ultrasound system 100 and other components included in or connected with portable ultrasound system 100. For example, control signals may be provided to an ultrasound probe (e.g., controlling the ultrasound emissions of the probe) and data may be received by ultrasound system 100 from the probe (e.g., imaging data).

In various embodiments, various ultrasound imaging systems may be provided with some or all of the features of the portable ultrasound system illustrated in FIGS. 1A-1B and -2. In various embodiments, various ultrasound imaging systems may be provided as a portable ultrasound system, a portable ultrasound transducer, a hand-held ultrasound device, a cart-based ultrasound system, an ultrasound system integrated into other diagnostic systems, etc.

### B. Systems and Methods for Pulsed Wave Predictive Processing

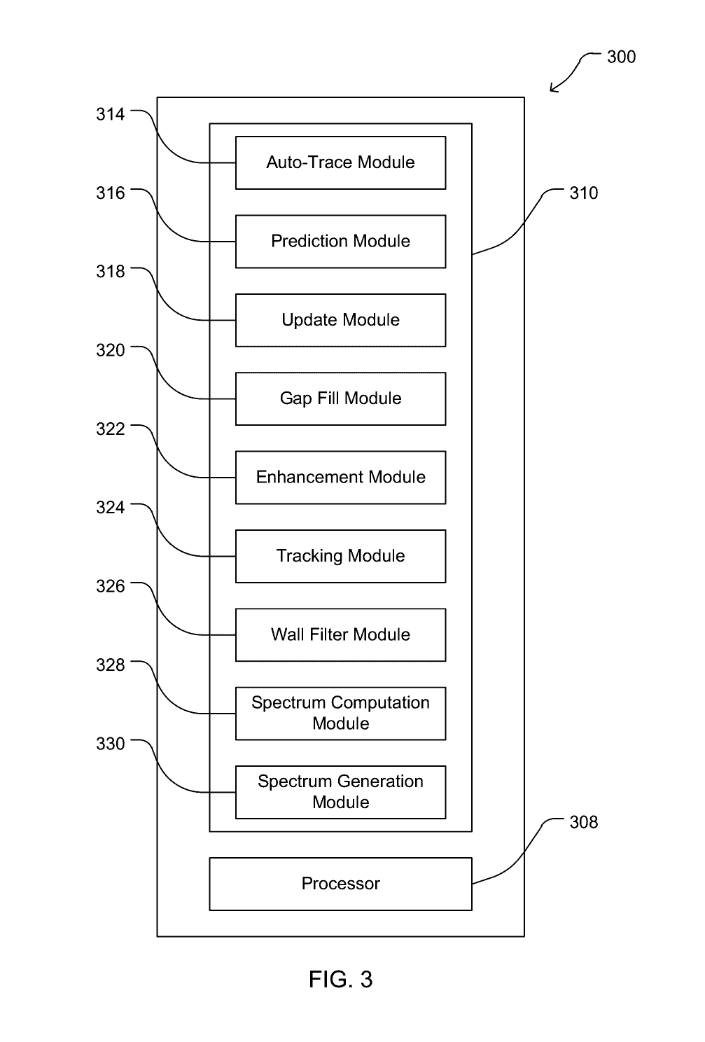

Referring now to FIG. 3, an embodiment of a processing circuit of an ultrasound system (e.g., ultrasound system 100) is illustrated. The processing circuit 300 includes a memory 310 and a processor 308. The processing circuit 300 can be similar to and perform similar functions as the processing circuit 210 described herein with reference to FIG. 2. For example, the memory 310 can be similar to the memory 212, and the processor 312 can be similar to the processor 214. As described herein with reference to FIG. 3, the processing circuit 300 (and particularly, memory 310 thereof) can include various electronic modules, circuits, or engines (e.g., prediction module 314, etc.), configured to execute various functions performed by an ultrasound system; in various embodiments, the processing circuit 300 can be organized in various ways for determining how functions are executed. The modules can be configured to share responsibilities by sending instructions to each other to execute algorithms and other functions, and receiving outputs generated by the module receiving the instructions. The modules illustrated in FIG. 3 may be interchanged in terms of the order in which corresponding actions are executed. For example, the enhancement module 322 and tracking module 324 can each be executed before or after the spectrum computation module 328 is executed.

The processing circuit 300 is configured to receive ultrasound information from an ultrasound transducer (e.g., an ultrasound transducer similar or identical to ultrasound transducer assembly 102). The ultrasound information can correspond to or represent blood flow of a patient. The ultrasound information can be received as being organized into ultrasound data samples. An ultrasound data sample can be raw data from the ultrasound transducer. For example, the ultrasound data sample can be an analog radio frequency signal outputted by the ultrasound transducer, or a digital data signal resulting from processing of the analog radio frequency signal by an analog-to-digital converter. The ultrasound data sample can represent a velocity of blood at a single point or within a region in space in the patient.

The ultrasound data sample can correspond to individual points of ultrasound information (e.g., a single point corresponding to amplitude, frequency, time, and/or position information; a single point corresponding to a velocity and time pair), or can be organized into segments corresponding to durations of time, such as durations of time corresponding to a heart cycle of a patient (e.g., sequences of points corresponding amplitude, frequency, time, and/or position information; sequences of points corresponding to velocities paired with times of a heart cycle of a patient). For example, an ultrasound data sample can include a sequence of data point pairs (e.g., raw data) of [frequency, time] corresponding to a heart cycle; or, if a Doppler equation algorithm has been executed to process the raw data, the ultrasound data sample can include a sequence of data point pairs of [velocity, time] corresponding to a heart cycle, or any other sequence of data point pairs corresponding to a Doppler spectrum based on the ultrasound information.

The processing circuit 300 can be configured to identify heart cycles of the ultrasound information (e.g., raw data received from the ultrasound transducer assembly 102) or of the waveform. The processing circuit can identify heart cycles based on time data received with or as part of the ultrasound information. For example, where the ultrasound information is received as ultrasound data samples of a defined sample length (e.g., defined duration of time), the processing circuit 310 can identify heart cycles based on their correspondence to each ultrasound data sample. This may be effective where the pulse repetition frequency or other parameter determining the sample length of the ultrasound samples corresponds to a heart cycle duration.

In some embodiments, the processing circuit 300 is configured to identify heart cycles based on a feature of the ultrasound information or the waveform. For example, the processing circuit 300 can distinguish heart cycles based on periodic features of heart cycles, such as peaks or troughs.

In some embodiments, the processing circuit 300 includes an auto-trace module 314. The auto-trace module 314 is configured to generate a waveform (e.g., a first waveform) by automatically tracing the ultrasound information. For example, the auto-trace module 314 can receive the ultrasound information, identify a feature of the ultrasound information, and generate the trace based on a value associated with the feature over time. The feature may be one or more of a mean velocity, a maximum velocity, a shape of the spectrum, an envelope of the spectrum, a power of the spectrum, and/or a pattern in such features or a pattern indicating whether the anatomy being tracked is moving, changing in conformation, moving out of the plane being tracked, or otherwise changing in state. The auto-trace module 314 can generate the waveform by following a feature of the ultrasound information over time. The waveform can include the ultrasound information over time (e.g., sequences of frequency vs. time points or velocity vs. time points), or can include an auto-traced feature of the ultrasound information over time (e.g., envelope over time, mean velocity over time, etc.). While some embodiments described herein generally refer to processing the auto-traced waveform for generating updated visual representations of current heart cycles, in various other embodiments, the raw data from the ultrasound transducer assembly 102 may also be used.

The auto-trace module 314 can be configured to generate the waveform based on an anatomy or anatomical state of the patient. For example, the auto-trace module 314 can generate the waveform based on a change or evolution of the anatomy, such as breathing or pulsation. The auto-trace module 314 can identify anatomical motion (e.g., valves opening or closing, vessels moving with gross body movements of the patient) and generate the waveform based on the anatomical motion. Various processes for tracking anatomical movement are described further herein with reference to the tracking module 324; similarly, tracked anatomical movement can be used by the auto-trace module 314 as a parameter for generating the waveform.

The auto-trace module 314 can be configured to execute auto-trace algorithms that identify traced features of ultrasound information. For example, the auto-trace module 324 can extract a traced shape corresponding to velocity and/or amplitude of velocity as a function of time of the ultrasound data samples. In some embodiments, tracing ultrasound information includes identifying velocity values in the ultrasound information, and interpolating velocities between consecutive velocity values (e.g., linearly interpolating between velocity values). The auto-trace module 314 can compute an envelope of the ultrasound data signal in the received Doppler spectrum. The auto-trace module 314 can be configured to continuously (e.g., automatically) extract traced shapes of velocity profiles of the ultrasound data samples. The auto-trace module 314 can store a template of a velocity profile (or traced shaped) of a heart cycle, or retrieve the template from another module of the memory 310, and group sequences of velocity and time data point pairs into ultrasound data samples corresponding to heart cycles. For example, the template can indicate expected locations of features such as peaks (e.g., points with increases in velocity prior to the point and decreases in velocity after the point), plateaus (e.g., points with relatively little change in velocity), increases in velocity, decreases in velocity, and/or troughs (e.g., points with decreases in velocity prior to the point and increases in velocity after the point) in a heart cycle, and the auto-trace module 314 can be configured to group sequences of velocity and time data point pairs to align with the expected locations of the features.

The processing circuit 300 can include a prediction module 316. The prediction module 316 is configured to predict, determine, or otherwise generate a waveform (e.g., second waveform, predicted waveform) based on the plurality of prior heart cycles. The predicted waveform can be an extrapolation of the plurality of prior heart cycles that indicates trends, changes, or other dynamic behavior of the subject from which the ultrasound information is detected. For example, the predicted waveform can account for trends or patterns in the plurality of prior heart cycle. The predicted waveform (or at least a portion thereof) can correspond to a time frame of the current heart cycle, such that a one-to-one comparison may be made between the current heart cycle and the predicted waveform (or the matching portion thereof). By generating a predicted waveform, the processing circuit 300 can accurately distinguish signal from noise in the ultrasound information by emphasizing the display of ultrasound information which follows trends or patterns represented in the predicted waveform. Using the predicted waveform to update or modify a visual representation of the current heart cycle, as will be described further herein, can improve the operation of an ultrasound system, including how the ultrasound information is displayed to a medical professional by enhancing the aesthetic value and accuracy of the information displayed.

The prediction module 316 can be configured to determine a pattern or trend of a feature of the plurality of prior heart cycles, and generate the predicted waveform based on the pattern or trend. The feature may be one or more of shapes, envelopes, mean velocity, maximum velocity, or power of the prior heart cycles. For example, the prediction module 316 can determine that the maximum velocity is increasing (or decreasing) over time, and generate the predicted waveform to have a maximum velocity determined based on the trend of increasing (or decreasing) maximum velocity over time. The prediction module 316 can determine that a heart rate of the patient is increasing (or decreasing) over time (e.g., based on a change in a periodic feature of the heart cycles such as a peak-to-peak time), and modify a period of time used to determine heart cycles based on the heart rate of the patient. The prediction module 316 can similarly recompute periods of time used to determine sample length, such as for recomputing spectra associated with a current heart cycle when the update module 318 updates a visual representation of the current heart cycle based on the predicted waveform.

In some embodiments, the prediction module 316 is configured to generate the predicted waveform based on an anatomical model of the patient. The anatomical model may indicate dynamic changes in the anatomy of the patient (e.g., valves opening/closing). The anatomical model may include or reference at least one of a mechanical model or a structural model of the anatomy of the patient, such as to more accurately represent a dynamically changing flow state of the patient to predict the flow state of the patient.

In some embodiments, the processing circuit 300 includes an update module 318. The update module 318 is configured to update a visual representation of the current heart cycle based on the predicted waveform. The visual representation may be updated at various stages during the processes described herein, prior to spectrum estimation or generation by the spectrum generation module 330 (e.g., before or after wall-filtering by the wall-filter module 326).

The update module 318 can be configured to determine a similarity (or a signal match) of the current heart cycle to the predicted waveform. In some embodiments, a relatively high similarity may indicate that the predicted waveform accurately represents anatomical behavior of the patient, such that information from the predicted waveform can be used to accurately improve the signal to noise ratio of the visual representation of the current heart cycle (as well as to fill in gaps or spatially enhance the visual representation of current heart cycle). In some embodiments, a relatively low similarity may indicate that the predicted waveform does not accurately represent anatomical behavior of the patient, such that the current heart cycle may need to be recomputed, such as by filling in gaps or moving a gate location of the current heart cycle; if a similarity or match is still not determined after the recomputation, the current heart cycle may be displayed without including features of the predicted waveform.

The update module 318 can be configured to determine the similarity by executing at least one of a sum of absolute differences algorithm, a cross-correlation algorithm, or a template matching algorithm. The sum of absolute differences algorithm can be executed by determining an absolute difference between corresponding points in time for the current heart cycle and the predicted waveform, and determining a sum of the absolute differences.

The update module 318 can execute the cross-correlation algorithm by executing a sliding dot product algorithm in order to measure similarity of the current heart cycle to the predicted waveform; as the result of the sliding dot product algorithm increases in magnitude, the similarity will increase, and vice versa.

The update module 318 can compare the current heart cycle to the predicted waveform based on a template matching algorithm. For example, the update module 318 can include a database storing a template of an ultrasound heart cycle or waveform, such as a template identifying common features of heart cycles such as velocity information as a function of time. For example, the template can include velocity information as a function of time corresponding to a template or expected heart cycle of the patient. In some embodiments, the template is a template of an auto-traced waveform. In some embodiments, the template is a non-dimensional shape of velocity for a heart cycle (e.g., expected velocity magnitudes or amplitudes for each point in time during the heart cycle, normalized to a scale such as a −100 to 100 scale); the template can be multiplied by a physiological parameter such as a flow state parameter (e.g., flow rate, flow velocity, etc.) to dimensionalize the template or otherwise apply the template to the patient and the patient's blood flow.

In some embodiments, the update module 318 is configured to update the visual representation of the current heart cycle based on whether the similarity exceeds one or more threshold values. For example, the update module 318 can be configured to compare the similarity to a first threshold (e.g., a relatively low threshold value). If the similarity exceeds the first threshold, the visual representation may be updated to include features of the predicted waveform. The update module 318 can also be compared to a second threshold (e.g., a medium threshold value that is greater than the low threshold value), and update the visual representation to include more features, or a greater share of features, of the predicted waveform.

For example, if the similarity is greater than the first threshold and less than or equal to the second threshold, features of the predicted waveform can be included by linear blending or alpha blending, where the update module 318 outputs the visual representation as output=(1−alpha)*(predicted waveform)+alpha*(current heart cycle), where alpha is a blending coefficient or proportionality coefficient that may correspond to an anatomical state of the patient. Alpha can be a proportionality constant determined as (alpha=0 if similarity is less than the first threshold; alpha=alpha_{low}, or is proportional to the similarity, if similarity is greater than or equal to the first threshold and less than the second threshold. Alpha may be adapted to an anatomical state of the patient, such as a flow velocity or flow strength, such that for low flow velocities, alpha is relatively greater (e.g., more of the predicted waveform is used), while for high flow velocities, alpha is relatively lesser (e.g., less of the predicted waveform is used). By adapting alpha to the anatomical state of the patient, the visual representation of the current heart cycle can be improved by more realistically accounting for how dynamic the blood flow is.

The update module 318 can modify the current heart cycle based on a flow state by altering a ratio of the predicted waveform included in or combined with the current heart cycle. For example, if the flow velocity is relatively low, or if the flow rate is relatively low, the current heart cycle can be modified to include more of the predicted waveform; if the flow velocity is relatively high, or if the flow rate is relatively high, the current heart cycle can be modified to include less of the predicted waveform. In some embodiments, such a ratio of the current heart cycle to the predicted waveform can be proportional to a ratio of a flow state of the current heart cycle to a flow state of the predicted waveform. For example, the modification of the current heart cycle can be dynamically adapted based on the flow state. In some embodiments, the thresholds are determined based on user input (e.g., user input received at user interfaces as described herein with reference to FIG. 2).

In some embodiments, if the similarity is greater than the second threshold, then the update module 318 can select one of the current heart cycle or the predicted waveform based on a selection factor representing the blood flow. This may be the case where the current heart cycle and the predicted waveform are very similar, such that any difference between the current heart cycle and the predicted waveform may be of a similar magnitude as noise information that distinguishes the current heart cycle from a true representation of the blood flow. For example, the selection factor may be a signal strength of the predicted waveform or the current heart cycle; if the current heart cycle has a greater signal strength than the predicted waveform, the current heart cycle is selected, otherwise the predicted waveform is selected. The signal strength selection may be based on an offset that biases the selection of the current heart cycle. For example, if the signal strength of the current heart cycle is at least a threshold fraction of the signal strength of the predicted waveform (e.g., at least 60%; at least 80%), then the current heart cycle is used, otherwise the predicted waveform is used. The selection factor may also be a maximum velocity. By using the strength or velocity as a selection factor, the visual representation of the current heart cycle is improved because the displayed spectrum can be more uniform over the display interval.

The update module 318 can be configured to modify the current heart cycle based on a nonlinear function. For example, the update module 318 can use an exponential function, a power law function, or any other nonlinear function to determine the proportion of predicted waveform to be included when modifying the current heart cycle. In some embodiments, the update module 318 is configured to modify the current heart cycle by nonlinearly increasing a portion of the predicted waveform combined with the current heart cycle as the similarity increases. In various embodiments, various functions and thresholds can be combined. For example, no data from the predicted waveform could be included if the similarity is less than a first threshold; the amount of data from the predicted waveform used could increase linearly as the similarity increases from the first threshold to the second threshold; the amount of data from the predicted waveform used could increase exponentially as the similarity increases from the second threshold to a maximum value (e.g., a maximum value indicating that the first characteristic and the second characteristic are identical).

In some embodiments, the processing circuit 300 includes a gap fill module 320. The gap fill module 320 can be configured to fill gaps in ultrasound information (e.g., an analog signal or a digital signal generated by sampling the analog signal) received from the ultrasound transducer assembly 102. Gaps in ultrasound information may occur during points in time in which ultrasound data is not acquired, such as due to limitations in the spatial range of ultrasound transducers, or dynamic action of an anatomy of the patient such as valves opening or closing. The gap fill module 320 can be configured to extract information from the predicted waveform to fill the gap, such as by aligning a heart cycle in the predicted waveform with the current heart cycle (e.g., aligning based on time, or based on duration from a common feature such as a peak velocity or peak frequency), and filling in the gap using corresponding or matching portions of the predicted waveform and the current heart cycle. The gap fill module 320 can be configured to identify a gap in the ultrasound information or the plurality of prior heart cycles, and predict a current gap in the current heart cycle based on the identified gap, such as by determining a pattern of the identified gap as it passes through the plurality of prior heart cycles (e.g., a continuous pattern, such as movement of the gap; a periodic pattern, such as the gap coming in and out of heart cycles).

The gap fill module 320 can fill the gaps using at least one of a pulse repetition frequency, a time extent for displaying B-mode images, or a current velocity. For example, one or more of such factors may be used to identify the gaps and/or predict the gaps in the predicted waveform. The pulse repetition frequency may account for whether the appearance of the gap in the ultrasound information is synchronous or asynchronous with the duration of heart cycles. The current velocity may indicate how a gap moves over time.

In some embodiments, the processing circuit 300 includes an enhancement module 322. The enhancement module 322 can be configured to perform spatial enhancement of the visual representation of the current heart cycle. The spatial enhancement may be determined based on the predicted waveform or features thereof, such as velocity or strength. The enhancement module 322 can be configured to use the predicted waveform to filter signal information from noise information in the ultrasound information, and spatially filter the noise information (e.g., filter based on frequency) to more clearly indicate the signal information. For example, by extrapolating or determining a trend or pattern of known signal information in the plurality of prior heart cycles, the enhancement module 322 can have greater certainty as to a difference between signal information and noise information.

The enhancement module 322 can be configured to enhance an edge or boundary of the visual representation of the current heart cycle. For example, the visual representation may include information associated with a plurality of detected frequencies or velocities at each point in time. In existing systems, it may be difficult to distinguish detected frequencies or velocities that correspond to signal information from noise. The enhancement module 322 can be configured to identify a boundary or edge of the waveform (e.g., a predicted waveform). The boundary or edge may be identified based on a known or predicted peak frequency or velocity of the waveform. The peak frequency or velocity of the current heart cycle may be compared to the peak frequency or velocity of the predicted waveform, and a similarity comparison may be used to update the edge or boundary of the visual representation of the current heart cycle. When generating the visual representation, the enhancement module 322 can enhance the edge or boundary by making the edge or boundary more clear, such as by increasing a brightness of the edge or boundary when displayed, or increasing a contrast ratio between the edge or boundary and other portions (e.g., a background portion) that may be displayed adjacent to the edge or boundary. The enhancement module 322 can filter (e.g., spatially filter, spatially locally enhance) a region near the edge or boundary to separate signal information from noise.

In some embodiments, the enhancement module 322 is configured to determine a filter direction based on the plurality of prior heart cycles, such as based on a trend of the peak frequency or velocity. The filter direction may indicate an expected or predicted direction for movement of the edge or boundary. The enhancement module 322 can filter the edge or boundary from noise information based on the filter direction. For example, if the plurality of prior heart cycles, or a predicted waveform based on the plurality of prior heart cycles, indicates that the peak velocity is decreasing, then the filter direction may be set to a decreasing direction. The enhancement module 322 can filter the edge or boundary to spatially enhance the edge or boundary more accurately by using the filter direction to predict changes to the edge or boundary.

In some embodiments, the processing circuit 300 includes a tracking module 324. The tracking module 324 is configured to adjust, update, move, or otherwise modify a sample volume of the ultrasound information, such as a gate (e.g., a sample volume) used to isolate a portion of ultrasound information of interest. The gate may be represented by a size and location, which can be controlled based on range and angle (e.g., azimuth) parameters used to extract waveforms from the ultrasound information. The tracking module 324 can track a plurality of gates, such as for matching one or more of the plurality of gates to the current heart cycle or the predicted waveform, or form tracking anatomy (e.g., using tracking module 324) corresponding to the plurality of gates. The tracking module 324 can be configured to detect a signal match between the current heart cycle and the predicted waveform as the tracking location is adjusted.

The tracking module 324 can be configured to track an anatomy of the patient based on expected motion. For example, the tracking module 324 can execute an expected motion algorithm to predict anatomical movement, and move the tracked anatomy (e.g., adjust the gate) based on the predicted movement. The tracking may be done continuously or periodically based on expected motion. The tracking module 324 may receive a user input indicating anatomical information associated with the expected motion, and executed the expected motion algorithm further based on the anatomical information.

In some embodiments, the tracking module 324 is configured to adjust the gate based on a comparison between the predicted waveform and the current heart cycle. For example, depending on a difference between the predicted waveform and the current heart cycle, the tracking module 324 can modify the range and/or angle to select new gate locations and sizes, and thus focus on new portions of the ultrasound information that may more accurately match dynamic activity of the subject being tracked by the ultrasound transducer assembly 102. In some embodiments, the range and/or angle may be modified proportional to the difference (e.g., a greater difference may indicate that an appropriate gate location is further away from the current gate location).

In some embodiments, the processing circuit 300 is configured to execute at least one of the gap fill module 320, the enhancement module 322, or the tracking module 324 based on a similarity of the current heart cycle to the predicted waveform, such as described with reference to the update module 318. For example, if the similarity is less than a threshold (e.g., a relatively low threshold value), it may be likely that gaps or movement in anatomy account for the dissimilarity between the current heart cycle and the predicted waveform. Filling in the gaps, or tracking the anatomy by changing gate location and size to identify a new location for spectrum determination may thus improve the operation of the ultrasound system. Such actions may be performed automatically.

The tracking module 324 can be configured to vary the gate location and size (e.g., vary the range, vary the angle or azimuth) to increase the similarity between the current heart cycle and the predicted waveform. For example, an search algorithm or optimization algorithm may be executed that varies at least one of the range or angle to increase or optimize the similarity, such as to search for a high similarity match, or the best match, between the current heart cycle and the predicted waveform. The tracking module 324 may alternatively or additionally execute a signal to noise ratio determination algorithm to determine a measure of signal to noise ratio that may change as the gate location and size are varied, such as to increase or optimize the signal to noise ratio when searching for a high similarity match, a high signal to noise ratio match, and/or a best match between the current heart cycle and the predicted waveform.

The processing circuit 300 can include a wall filter module 326. The wall filter module 320 is configured to filter the ultrasound information to remove features corresponding to walls of blood vessels of the patient. For example, the wall filter module 320 can be configured to identify and remove low-frequency components in the ultrasound information detected by the ultrasound transducer assembly 102, such as by applying a high pass filter to the ultrasound information. The high pass filter can be calibrated based on stored information regarding typical frequencies detected for blood flow, as compared to typical frequencies detected for blood vessel walls The high pass filter can be calibrated dynamically and/or in response to user input, such as user input indicating feedback from a user describing whether the displayed spectrum of the ultrasound data samples includes information representative of blood vessel walls. The wall filter module 320 can be interchangeable with the gap fill module 320.

The processing circuit can include a spectrum computation module 328. The spectrum computation module 322 can be configured to generate Doppler spectrum of ultrasound data samples or ultrasound information. The spectrum computation module 322 can receive the ultrasound information as Doppler frequency shifts detected by the ultrasound transducer assembly 102, and process the Doppler frequency shifts by executing a Doppler equation algorithm to determine velocity information (e.g., determine velocity information in the time domain, determine velocity information as a function of time and/or space, etc.). In some embodiments, the spectrum computation module 328 is configured to process the ultrasound information to identify frequency shifts prior to executing a Doppler equation algorithm to determine velocity information. The spectrum computation module 328 can be configured to output velocity information as paired points (e.g., [velocity, time] pairs).

The processing circuit 300 can include an spectrum generation module 330. The spectrum generation module 330 is configured to generate an ultrasound spectrum or image (e.g., spectrum data corresponding to an ultrasound spectrum and/or image data corresponding to an ultrasound image) based on the visual representation of the current heart cycle, and can output the ultrasound spectrum in a format for display (e.g., for display by touchscreen 172, main display 190, etc.). The spectrum generation module 330 can output the ultrasound spectrum via display interface 240. The spectrum generation module 330 can generate an ultrasound spectrum including an array or matrix of pixels, each pixel corresponding to a displayed point on a display. The spectrum generation module 330 can include color and brightness information with each pixel (e.g., color and brightness information corresponding to an ultrasound data sample to be displayed using one or more pixels). The spectrum generation module 330 may be included in dedicated graphics processing electronics (e.g., a graphics processing unit).

In some embodiments, the spectrum generation module 330 is configured to generate duplex (and/or triplex) spectrum information for display. For example, the spectrum generation module 330 can generate an ultrasound spectrum or image (or multiple ultrasound spectra or images to be displayed adjacent to one another, superimposed or overlaid, or otherwise coordinated) with a first portion corresponding to a structure of the patient's body (e.g., a two-dimensional image of the structure) and a second portion corresponding to the visual representation of the current heart cycle (e.g., corresponding to blood flow information). For example, the spectrum generation module 330 can be configured to use the visual representation of the current heart cycle output by the update module 318 to determine colors for displaying blood flow (e.g., using red to indicate blood flow in a first direction, blue to show blood flow in a second direction, and wavelengths within a red wavelength range (e.g., approximately 620-780 nm) or blue wavelength range (e.g., approximately 455-490 nm) to show magnitude of blood flow).

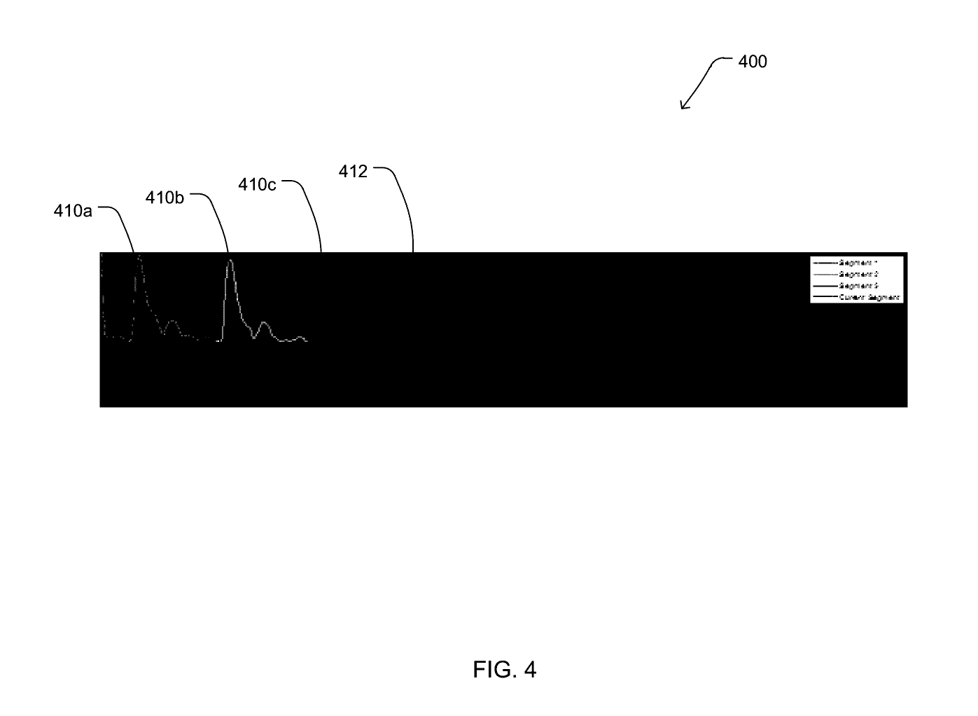

Referring now to FIG. 4, an embodiment of an ultrasound spectrum 400 displaying blood flow velocity information is shown. The ultrasound spectrum 400 includes a plurality of prior heart cycle data samples 410a, 410b, 410c corresponding to ultrasound information detected prior to a ultrasound information of a current heart cycle data sample 412. The data samples 410a-410c, 412 can indicate velocity and time information of blood flow of the patient. An ultrasound system (e.g., ultrasound system 100, an ultrasound system including processing circuit 300, etc.) can be configured to update the current heart cycle 412 based on a predicted waveform generated based on the prior heart cycle data samples 410a, 410b, 410c. For example, depending on the similarity of the predicted waveform to the current heart cycle, data of the predicted waveform can be included when displaying the current heart cycle data sample 412. As such, the signal to noise ratio of the current heart cycle 412 is improved by accounting for dynamic changes to the blood flow of the patient using the predicted waveform.

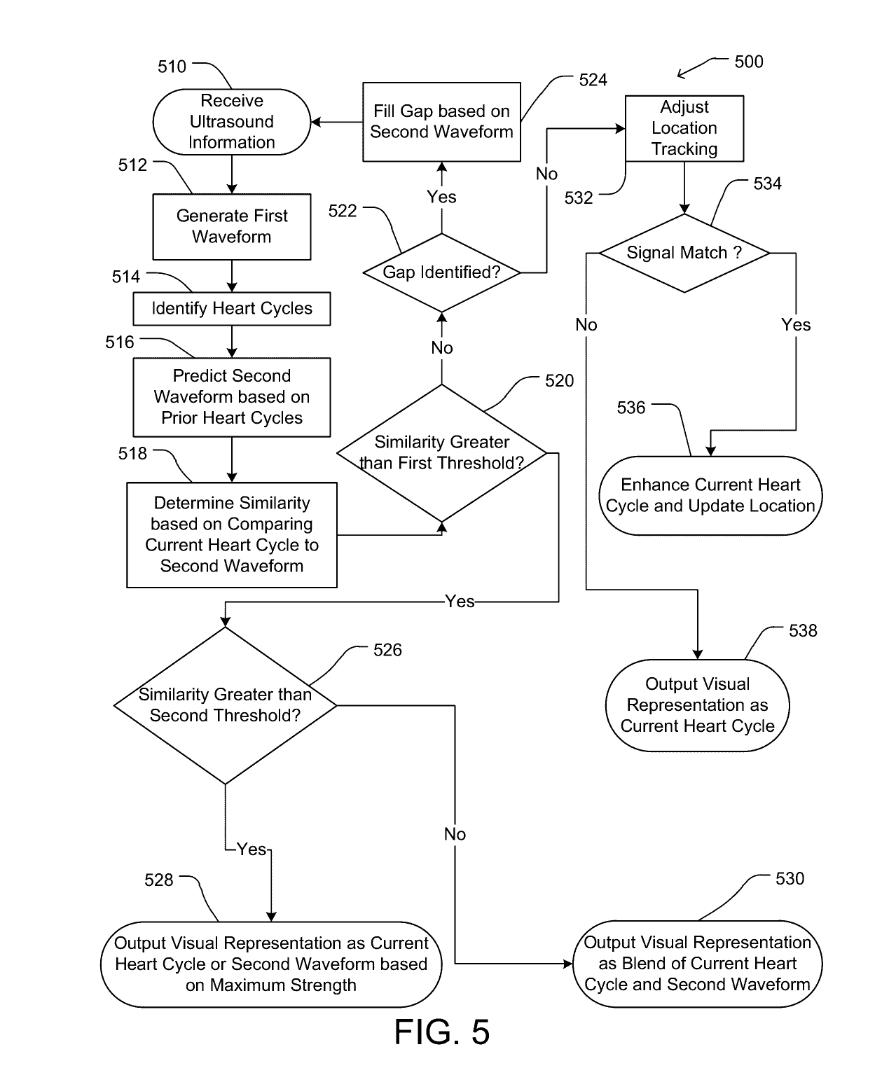

Referring now to FIG. 5, a method 500 of predictively generating a visual representation of ultrasound information is shown according to some embodiments. The method can be implemented by an ultrasound system, such as ultrasound system 100, an ultrasound system including processing circuit 300, etc. The method 500 can be performed for displaying an ultrasound spectrum or image to a user performing an ultrasound diagnostic procedure.

At 510, ultrasound information is received. For example, an ultrasound transducer probe can be positioned adjacent to the patient to detect ultrasound information from the patient.

At 512, a first waveform is generated. The first waveform may be generated to capture features of the ultrasound information, such as a shape, envelope, velocity profile (e.g., a distribution of velocities, a mean or maximum velocity), or power of the ultrasound information. The first waveform may be generated by executing an auto-trace algorithm to trace the waveform from the ultrasound information. The first waveform may also be raw data from the ultrasound information.

At 514, a plurality of prior heart cycles and a current heart cycle are identified from the first waveform. The heart cycles may be identified based on anatomical information regarding the patient, including heart rate, or by identifying periodic features of the first waveform, such as peak velocities or peak frequencies.

At 516, a second waveform is predicted based on the plurality of prior heart cycles. The second waveform may be an extrapolation of the plurality of prior heart cycles. The second waveform may be based on a trend or pattern identified in the plurality of prior heart cycles, such as a trend or pattern of strength, velocity, or shape.

At 518, a similarity is determined based on comparing the current heart cycle to the second waveform. The similarity may be determined based on executing at least one of a sum of absolute differences algorithm, a cross-correlation algorithm, or a template matching algorithm. The similarity may indicate how closely the second waveform represents an anatomical state of the patient as compared to the current heart cycle.

At 520, the similarity is compared to a first threshold (e.g., a relatively low threshold). If the similarity is not greater than the first threshold, then at 522, a gap may be identified in the current heart cycle. If a gap is identified in the current heart cycle, then at 524, the gap is filled in based on the second waveform. For example, a location of the gap may be predicted for the predicted waveform, and used to fill in the gap in the current heart cycle (e.g., features of the gap in the first waveform may be extrapolated to points in time corresponding to when the gap occurs in the current heart cycle, and corresponding features of the second waveform may be included in the current heart cycle to fill in the gap). After the gap is filled, new ultrasound information may be received.

If the similarity is greater than the first threshold, then at 526, the similarity is compared to a second threshold (e.g., a second threshold greater than the first threshold). If the similarity is greater than the second threshold, then at 528, the visual representation can be outputted as one of the current heart cycle or the predicted waveform having a greater signal strength, which may indicate the more accurate representation of the patient. If the similarity is greater than the first threshold but less than or equal to the second threshold, then at 530, the visual representation can be generated and outputted by blending the current heart cycle and the second waveform, such as by including features from the second waveform in the current heart cycle in a manner proportional to the similarity.

If a gap is not identified in a current location or in the current heart cycle, then at 532, location tracking may be adjusted. For example, based on the second waveform, a plurality of gate locations may be used to determine whether there is a more accurate or similar match between the current heart cycle and the second waveform. Similarly, multiple sample lengths for spectra or spectral lines may be used to adjust the location. If the adjusted tracking results in a new signal match (e.g., a relatively high similarity), then at 536, the current heart cycle is spatially enhanced (e.g., by applying a spatial filter at an edge or boundary of the current heart cycle), and the spatial location is updated to correspond to the location at which a signal match was determined. If the adjusted tracking does not result in a new signal match (e.g., a relatively low similarity), then at 538, the visual representation is outputted as the current heart cycle.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system, comprising:
an ultrasound transducer configured to detect ultrasound information from a patient and output the ultrasound information, the ultrasound information representing blood flow of the patient;
a processing circuit configured to:
generate a first waveform by automatically tracing the ultrasound information;
identify a plurality of prior heart cycles of the first waveform and a current heart cycle of the first waveform;
predict a second waveform based on the plurality of prior heart cycles; and
update a visual representation of the current heart cycle based on the second waveform; and
a display configured to display the visual representation of the current heart cycle.

2. The system of claim 1, wherein the processing circuit is configured to predict the second waveform based on a trend of a feature of the first waveform, wherein the feature includes at least one of a maximum velocity, a shape, a mean velocity, or a power of the waveform.

3. The system of claim 1, wherein the processing circuit is further configured to: predict a gap in the current heart cycle based on the plurality of prior heart cycles; and update the visual representation to fill in the gap based on the plurality of prior heart cycles.

4. The system of claim 3, wherein the ultrasound information includes at least one of spectral data or time data, and the processing circuit is configured to fill in the gap by predicting a pattern of the at least one of the spectral data or time data, calculating a difference between the pattern and the current heart cycle, and updating the visual representation of the current heart cycle based on the difference.

5. The system of claim 1, wherein the processing circuit is further configured to enhance a boundary between the visual representation of the current heart cycle and a background portion based on the second waveform.

6. The system of claim 5, wherein the processing circuit is configured to enhance the boundary by increasing a contrast ratio between the visual representation of the current cycle and the background portion.

7. The system of claim 5, wherein the processing circuit is further configured to filter a portion of the ultrasound information in a vicinity of the boundary based on a filter direction, the filter direction determined based on a trend of the plurality of prior heart cycles.

8. The system of claim 1, wherein the processing circuit is further configured to determine a similarity between the current heart cycle and the plurality of prior heart cycles.

9. The system of claim 8, wherein the processing circuit is further configured to compare the similarity to a first threshold, and update the visual representation of the current heart cycle to include features of the second waveform if the similarity is greater than the first threshold.

10. The system of claim 8, wherein the processing circuit is further configured to compare the similarity to a first threshold, and if the similarity is less than a first threshold, adjust a location used to select the plurality of prior heart cycles, determine a third waveform based on the adjusted location, determine a second similarity between the third waveform and the current heart cycle, and update the location used to select the plurality of prior heart cycles if the second similarity is greater than a second threshold.

11. The system of claim 1, wherein the processing circuit is configured to predict the second waveform further based on an anatomical model of the patient.

12. The system of claim 1, wherein the processing circuit is configured to determine at least one of a pulse wave strength or pulse wave velocity of the first waveform, and update the visual representation of the current heart cycle further based on the at least one of the pulse wave strength or pulse wave velocity.

13. The system of claim 1, wherein the processing circuit is further configured to identify a plurality of prior gate locations based on the plurality of prior heart cycles, identify a current gate location based on the current heart cycle, determined a predicted gate location based on the plurality of prior gate locations, compare the predicted gate location to the current gate location, and update the visual representation of the current heart cycle based on the comparison.

14. The system of claim 13, wherein the processing circuit is further configured to track an anatomy of the patient based on the predicted gate location.

15. A method, comprising:
receiving ultrasound information representing blood flow of a patient from an ultrasound transducer;
generating a first waveform by automatically tracing the ultrasound information;
identifying a plurality of prior heart cycles of the first waveform and a current heart cycle of the first waveform;
predicting a second waveform based on the plurality of prior heart cycles;
updating a visual representation of the current heart cycle based on the second waveform; and
displaying the visual representation of the current heart cycle.

16. The method of claim 15, further comprising determining a trend of a feature of the first waveform, wherein the second waveform is predicted further based on the trend.

17. The method of claim 16, further comprising:
determining a filter direction based on the trend; and
filtering a portion of the ultrasound information at a boundary between the visual representation of the current heart cycle and a background portion based on the filter direction to enhance the boundary.

18. The method of claim 15, further comprising:
predicting a gap in the current heart cycle based on the plurality of prior heart cycles; and
updating the visual representation to fill in the gap based on the plurality of prior heart cycles.

19. The method of claim 15, further comprising:
determining a similarity between the current heart cycle and the second waveform;
comparing the similarity to a first threshold; and
updating the visual representation to include features of the second waveform if the similarity if the similarity is greater than the first threshold.

20. The method of claim 19, wherein predicting the second waveform includes predicting the second waveform based on an anatomical model of the patient.
